(19) United States
(12) Patent Application Publication
(10) Pub. No.: US 2016/0367411 A1
(43) Pub. Date: Dec. 22, 2016

(54) MALE URINARY INCONTINENCE DEVICE

(71) Applicants: William Justiz, Naples, FL (US); Christopher Carfi, Palo Alto, CA (US); Leif Chastaine, Meridian, ID (US)

(72) Inventors: William Justiz, Naples, FL (US); Christopher Carfi, Palo Alto, CA (US); Leif Chastaine, Meridian, ID (US)

(21) Appl. No.: 15/187,773

(22) Filed: Jun. 20, 2016

Related U.S. Application Data
(60) Provisional application No. 62/182,029, filed on Jun. 19, 2015.

(51) Int. Cl. A61F 13/471; A61F 13/64; A61F 13/60; A61F 13/56

(52) U.S. Cl. CPC A61F 13/471; A61F 13/5611; A61F 13/64; A61F 13/60

(57) ABSTRACT

A male urinary incontinence device shaped to be worn on a user's penis. The device includes a base member having proximal and distal ends, and upper and lower surfaces, as well as a pouch located at the distal end of the base member. A method of fabricating a male incontinence device is also provided.

### CROSS REFERENCE TO RELATED APPLICATIONS

This application claims priority to U.S. Provisional Patent Application No. 62/182,029, filed on Jun. 19, 2015, entitled “MALE URINARY INCONTINENCE DEVICE.” The entire disclosure of the foregoing provisional patent application is incorporated by reference herein.

### BACKGROUND

Incontinence is defined as the involuntary passage of urine. It is a common condition that can be the result of complications of medical disease or surgical procedures. Incontinence can have a profoundly negative impact upon quality of life, limiting daily activities and affecting psychological wellbeing, social interaction and interpersonal relationships. Incontinence often occurs in obese individuals, the elderly and following certain surgeries. The most common medical procedures in men that lead to incontinence is prostate irradiation or resection. In 2011, per the Centers for Disease Control (CDC), 209,292 men were diagnosed with cancer of the prostate. According to the Journal of European Urology October, 2013, pages 672-9 (pmid 23587870), 60% of elderly prostate cancer survivors reported urinary incontinence.

Some degree of incontinence occurs in nearly all male patients for the first 3-6 months after radical prostatectomy. Within a year after the procedure, most men regain continence, although some leakage may still occur. Also according to the Journal of Urology September, 2010, pages 1022-1027 (PMID 20643440), severe urinary incontinence was found in up to 16% of American males. Urinary leakage may be of such amount to seep through undergarments and clothing. Coping with leakage is a significant problem. Odors are another problem. Additionally, according to the article in Health Tech Assessment (PMID 18547500), diapers were found to be better than other current products on the market.

The above information proves this is a common problem with inadequate devices to contain urinary spillage. It also demonstrates a need for a new and novel approach to male urinary incontinence.

The current devices on the market can be broken down into three broad categories. Those categories are diapers, pads and tubes. Adult incontinence diapers are a variation of the product that American mothers have been placing on their babies for over 45 years. This style of product is demoralizing and uncomfortable for the adult male wearer. It is uncomfortable in that it is bulky particularly in the perineal region even when it is first worn. This worsens as the product fills with urine. The added weight and bulk of the urine lead to changes in walking and gait patterns. This can cause the product to shift, increasing the likelihood that leaks will occur. Furthermore, changes in gait patterns, particularly in the elderly, can stress arthritic joints and promote inflammation and pain. Finally, the diaper devices cover large areas with impermeable plastic. This can lead to fungal growth and skin breakdown due to moisture trapping.

The pad-style of product covers the penis in a fashion similar to a feminine menstrual pad. This device, like the diaper, is not discrete and can add to the social stigma the wearer experiences. This device also fails to prevent splashing of urine onto the pelvic skin. Protracted skin exposure to liquids can lead to irritation, skin breakdown, ulceration, and possibly fungal growth. For the active wearer, this device does not provide adequate coverage for a user who's penis shifts position during jogging, cycling, walking or other strenuous activities. Furthermore, should the wearer acquire an erection, this will lead to protrusion of the penis outside the border of the pad. When the flaccid state returns and with it incontinence, the device fails and the clothing becomes urine soaked. Additionally, trapping the penis between the device and the pelvic skin can be uncomfortable.

Existing tube structures are relatively inflexible and thick walled. This creates an obvious and unnatural bulge in the user's clothing. The bulge compromises the user's desire for discreteness. In an attempt to address the bulge problem, existing prior art device developers have used thinner materials to maintain flexibility; however, this leads to diminished absorbency. Also, the tube designs are typically held in place by straps at the base of the penis. If the wearer acquires an erection, the strap that was comfortable in the flaccid state becomes constricting and uncomfortable.

While a variety of devices and techniques may exist for providing male urinary incontinence devices, it is believed that no one prior to the inventors has made or used an invention as described herein.

### BRIEF DESCRIPTION OF THE DRAWINGS

While the specification concludes with claims that particularly point out and distinctly claim the invention, it is believed the present invention will be better understood from the following description of certain examples taken in conjunction with the accompanying drawings. In the drawings, like numerals represent like elements throughout the several views.

FIG. 1 depicts an embodiment of the male urinary incontinence device.

FIG. 2 depicts the device of FIG. 1, as worn by the user.

FIG. 3 is a top plan view of the laminated blank from which the device of FIG. 1 is fabricated.

FIGS. 4 and 5 depict the process of fabricating the device of FIG. 1 from the laminated blank.

FIG. 6 is a bottom plan view of the device of FIG. 1.

FIG. 7 is the same view as FIG. 3, wherein the acquisition layer is shown by broken line.

FIG. 8 is a cross-sectional view of the device of FIG. 7, taken along the line 8-8 thereof.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples should not be used to limit the scope of the present invention. Other features, aspects, and advantages of the versions disclosed herein will become apparent to those skilled in the art from the following description. As will be realized, the versions described herein are capable of other different and obvious aspects, all without departing from the invention. Accordingly, the drawings and descriptions should be regarded as illustrative in nature and not restrictive.

The devices shown and described herein provide a disposable, discrete, and comfortable device for absorbing inadvertent urinary leakage due to male urinary incontinence. The device accommodates the changing shape, position and size of the penis due to erections, activities, and shifting for comfort, while absorbing urine in order to protect clothing, provide comfort and prevent embarrassment arising from incidents of male incontinence.

One embodiment of the disposable male incontinence device disclosed herein comprises a base member having a openable pouch at one end thereof. The pouch has an opening that extends above the upper surface of the base member, and is configured to receive the glans (head) portion of a user's penis therein. The base member and pouch are formed of a laminated structure comprising: a bottom layer, a top layer, and a liquid absorbent core sandwiched between the top and bottom layers. The bottom layer is essentially impermeable to liquid, while the top layer is permeable to fluids—particularly urine. The upper surface of the base member is worn against the ventral side of a user's penis, with the glans of the penis located within the pouch such that any urine released from the user is absorbed through the top layer into the absorbent core where it is stored. The device is comfortable, thin, discreet and easy to wear. As the absorbent core collects urine, the moisture is pulled away from the penis, thereby reducing irritation, skin breakdown, ulceration, and fungal growth.

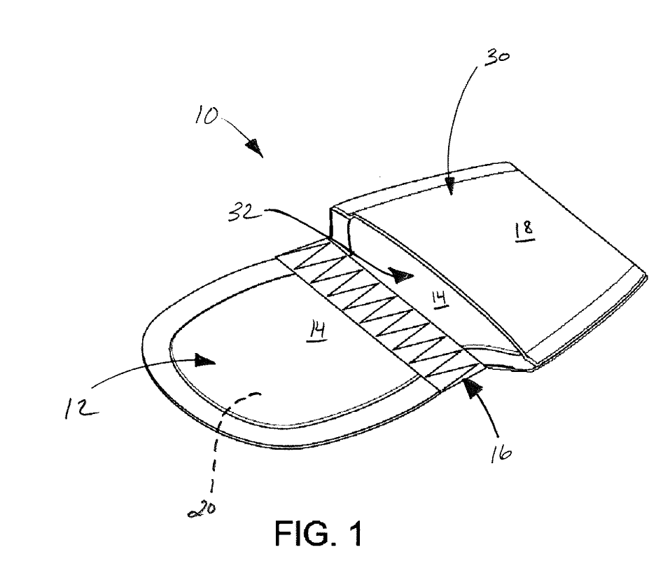

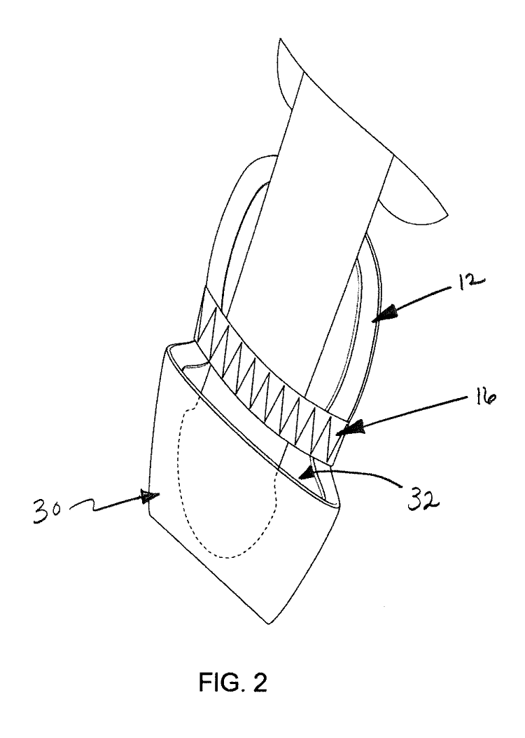

FIG. 1 depicts a disposable male incontinence device (10) comprising a base member (12) and a pouch (30) at the distal end of the base member (12). The pouch (30) includes an opening (32) that extends above the upper surface of the base member, and is configured to receive the glans (head) portion of a user's penis therein. An elastic strap (16) extends across the width of the base member (12), immediately adjacent the opening (32) of the pouch (30). As shown in FIG. 2, the strap (16) can be used to maintain the device (10) in position. The user inserts his penis under the strap (16) and into the pocket (30). The strap (16) maintains the tip of the penis inside the pocket as the penile shaft increases in circumference and/or shifts. As best seen in FIG. 1, the strap (16) lies flat against the upper surface of the base member (12) such that the incontinence device may be worn without using the strap, if desired. In an alternative embodiment, the strap (16) may be omitted entirely.

As used herein, “disposable” generally means that the device is not intended to be laundered or reused after a single use. The top layer (14) (sometimes referred to as a topsheet) is the layer intended to be worn against the user's penis (i.e., the body-facing surface). The bottom layer (18; see FIG. 8) (sometimes referred to as a backsheet) is intended to be worn against the user's garment, particularly the user's underwear (i.e., the garment-facing surface).

The top layer (14) is preferably compliant, soft feeling, and non-irritating to the user's skin. The top layer is also liquid pervious, permitting liquids (urine) to readily penetrate through its thickness. Suitable top layers may be manufactured from a wide variety of materials such as woven and nonwoven materials (e.g., a nonwoven web of fibers). Suitable woven and nonwoven materials can be comprised of natural fibers (e.g., wood or cotton fibers), synthetic fibers (e.g., polymeric fibers such as polyester, polypropylene, or polyethylene fibers) or from a combination of natural and synthetic fibers. When the top layer comprises a nonwoven web, the web may be manufactured by a wide number of known techniques. For example, the nonwoven may be spunbonded, carded, wet-laid, melt-blown, hydroentangled, combinations of the above, or the like. The top layer, e.g., comprising a soft non-woven sheet, can be plain or treated with a wide range of substances, such as aloe vera, vitamin E, shea butter, and the like.

The top and bottom layers sandwich the absorbent core (20) therebetween, as well as one or more intermediate layers. One particular intermediate layer employed in some embodiments of the incontinence device described herein is an acquisition and distribution layer (“ADL”) (22) disposed between the absorbent core (20) and the top layer (14) (see FIG. 8). As is known to those skilled in the art, the ADL (22) facilitates the absorption and distribution of urine by the absorbent core, allowing urine to be absorbed from the ADL (22) into the core (20). A conventional nonwoven ADL (14) is used in the devices described herein.

A variety of materials can be used for the absorbent core (20), including superabsorbent materials such as absorbent gelling materials (AGM), also referred to as superabsorbent polymers (SAP). Such materials are typically used in combination with absorbent hydrophilic fibers such as cellulose. One particularly type of absorbent core (20) is a combination of a polyacrylate based material, in particle form, in combination with cellulose.

The bottom layer (18) is essentially liquid impermeable, and may comprise any of a variety of materials known to those skilled in the art for use as diaper backsheets and the like. Such materials include various nonwoven materials that are essentially liquid impermeable. The bottom layer (18) prevents absorbed urine in the absorbent core from wetting articles which contact the bottom layer (18) such as the user's underwear. The bottom layer (18) can also be vapor permeable (“breathable”), while remaining liquid impermeable. In this instance, microporous plastic films are typically used which are water vapor permeable while remaining essentially impermeable to liquids.

As further discussed herein, an adhesive layer (24) may also be provided on the outer surface of the bottom layer (18), along with a removable release sheet (26) which is removed to expose the adhesive layer (24).

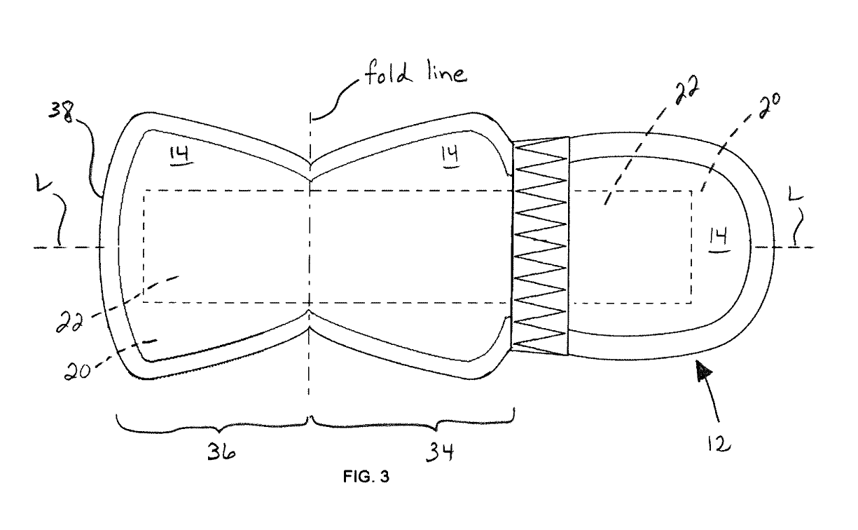

The base member and pocket can be formed in a variety of manners. In the particular embodiment shown, the pouch-forming components are integral with the base member (12). As best seen in FIG. 3, the base member (12) and pouch-forming components are provided as a unitary, multi-layer laminate structure. The pouch-forming components include a lower pouch section (34) and an upper pouch section (36). The lower pouch section (34) is located at the distal end of the base member (12), with the sides of the base member (12) flaring outwardly towards the proximal end of the lower pouch section (34). The sides of the lower pouch section (34) then taper inwardly toward the longitudinal axis (L) of the device to the distal end of the lower pouch section (34) which coincides with a fold line (see FIG. 3). The upper pouch section (36) is located at the distal end of the first pouch-section (34), with the sides of the upper pouch section (36) extending tapering outwardly away from the longitudinal axis (L) of the device to a curved distal end (38) of the upper pouch section (36). Thus, the sides of the upper pouch section (36) are a mirror-image of the sides of the lower pouch section (34). During fabrication, the upper pouch section (36) is folded over the lower pouch section (34), and the sides of the two pouch sections are adhered to one another (e.g., by heat sealing) so as to form pouch (30) between the upper and lower pouch sections.

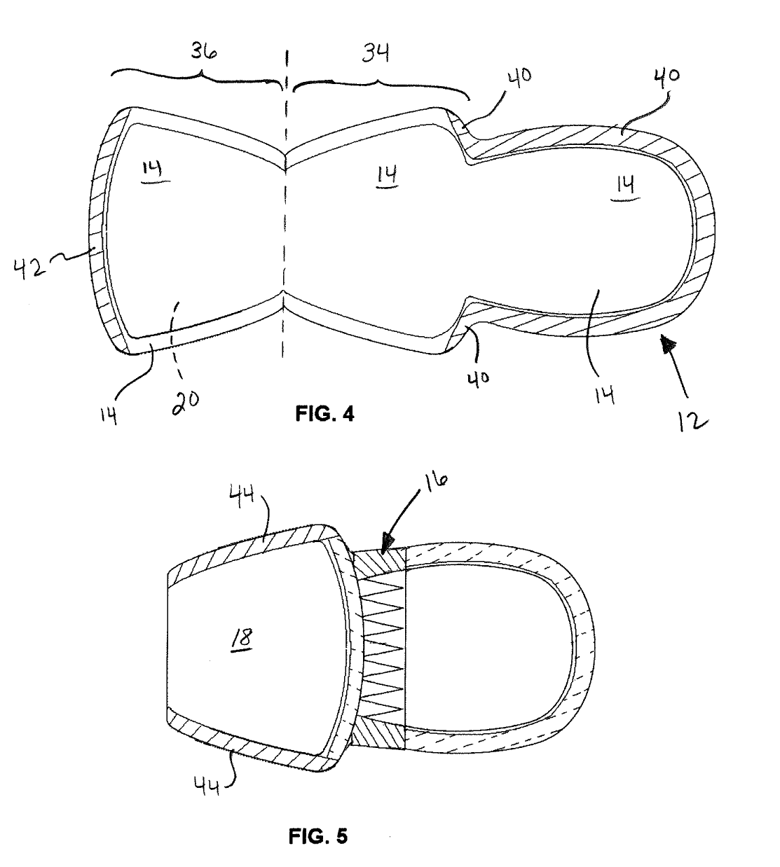

The fabrication process is best seen in FIGS. 4 and 5. First, the top and bottom layers (14, 18) are cut (e.g., die cut) from suitable sheets of material, and the absorbent core (20) and ADL (22; see FIG. 3) are located between the top and bottom layers, as shown. The top and bottom layers (14, 18) are sealed to one another about the periphery of base member (12) and the flared proximal ends of upper pouch section (34), at a border seal (40). Similarly, the top and bottom layers (14, 18) are also sealed to one another along curved distal end (38) of the upper pouch section (36) at a second border seal (42). The mating edges are sealed to one another using an adhesive, by heat sealing, a combination of an adhesive and heat sealing, ultrasonic welding, or any other method known to those skilled in the art that provides a water-tight seal. The border seals may or may not include the absorbent core (20). In the embodiment shown, the border seals are located immediately adjacent the periphery of the absorbent core (20). Thereafter, the elastic strap (16) is added by sealing the outer edges of the elastic strap to the top layer (14), as shown.

Next, the upper pouch section (36) is folded over the lower pouch section (34), and the sides of the two pouch sections are adhered to one another (e.g., by heat sealing) along border seals (44) so as to form pouch (30) between the upper and lower pouch sections. In the particular embodiment shown, the curved distal end (38) of the upper pouch section (36) extends partially over the strap (16). In other embodiments the distal end of the upper pouch section (36) completely covers the elastic strap (16), or, alternatively, does not cover any portion of the strap (16).

In some embodiments, the additional features may be included between the top and layer (14) and the absorbent core (20) to direct urine into the absorbent core for capture. For example, the ADL (22) (or another layer) can include apertures that that lead to channels which extend in a generally longitudinal direction from the end of the pocket towards the proximal end of the base member (12). Such channels have a lower permeability than the remaining portions of the ADL, and aid in directing urine towards the distal end of the device (i.e., away from pocket (30)) and away from the user's penis.

In still further embodiments, the device is configured such that the absorbent core (20) comprises a progressively more absorbent area from the proximal end of the base member (12) to the section inside the cowl. The area comprises a gradient of a first section of low-density material having a larger pore size at the cowl end progressing to a final section of high-density material at the proximal end (i.e., the end of base member (12). The gradient increases the rate of absorption of liquid at the pocket end and the amount of liquid stored at the opposite end. The absorbent core can comprise additional layers or additional material at the proximal end as compared to the pocket end in order to create the progressively more absorbent area. For example, the portion outside of the pocket can be configured to store about 20% to about 80% more liquid than the pocket portion of the device. This progression draws fluid away from the tip of the penis and the skin of the user. In one embodiment, the absorbent core (20) can absorb about 40 to 60 cc of urine, which covers mild to moderate incontinence.

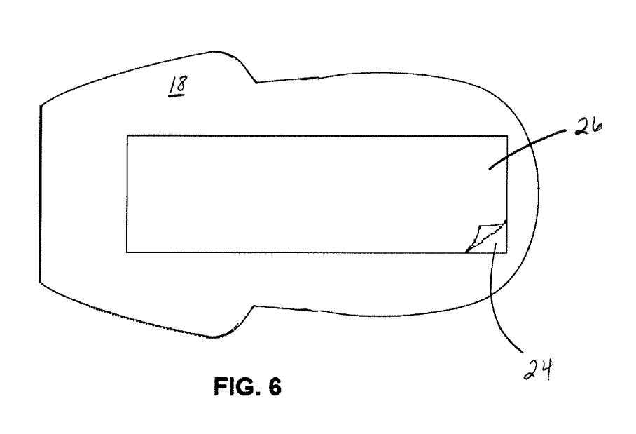

As mentioned previously, an adhesive layer (24) may also be provided on the outer surface of the bottom layer (18), along with a removable release sheet (26) which is removed to expose the adhesive layer (24). The adhesive layer can be used to releasably attach the device to the user's underwear (boxers or briefs) or other garment. The adhesive layer can extend across a portion of the base member (12) and a portion of the lower pouch section (34), but typically not the upper pouch section, as best seen in FIG. 6.

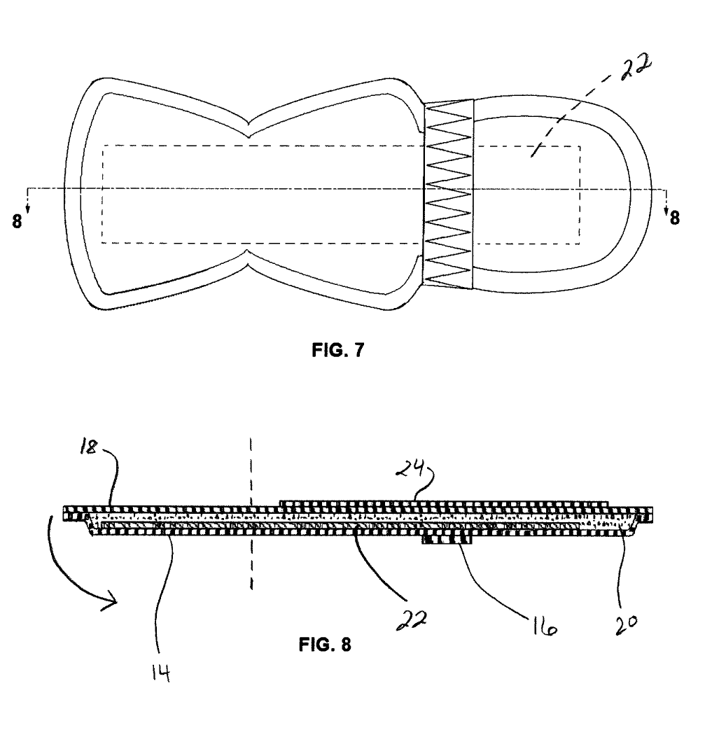

Since the pocket (30) is comprised of the same layers that make up the base member (12), the liquid permeable top layer (14) of the upper pouch section (36) faces the liquid permeable top layer (14) of the lower pouch section (34). Similarly, the liquid impermeable bottom layers (18) of the pocket (30) face outwardly. Thus, since the pocket (30) is configured to contain the tip of the user's penis, urine is not able to escape from the device to the user's undergarment. In addition, the ADL can extend across at least a portion of the length of the base member (12) and both the upper and lower pouch sections (34, 36), as seen in FIG. 7. Alternatively, the ADL can extend across at least a portion of the upper and lower pouch sections (34, 36).

In some embodiments, the overall length of the device is between 10 and 20 cm in length, with the interior width of the pocket between 4 and 10 centimeters. In some embodiments, the interior length of the pocket is between 30 and 60% of the overall length of the device. The device is also compact, and can even be folded for storage prior to use so that it fits inside a wallet. The device is also advantageous in that it can be comfortably and discreetly worn throughout the day and night, without becoming dislodged.

While several devices and components thereof have been discussed in detail above, it should be understood that the components, features, configurations, and methods of using the devices discussed are not limited to the contexts provided above. In particular, components, features, configurations, and methods of use described in the context of one of the devices may be incorporated into any of the other devices. Furthermore, not limited to the further description provided below, additional and alternative suitable components, features, configurations, and methods of using the devices, as well as various ways in which the teachings herein may be combined and interchanged, will be apparent to those of ordinary skill in the art in view of the teachings herein.

Having shown and described various versions in the present disclosure, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required.

What is claimed is:

1. A disposable male incontinence device comprising:
(a) a base member having proximal and distal ends, and upper and lower surfaces; and
(b) a pouch located at the distal end of the base member, said pouch having upper and lower interior surfaces, upper and lower exterior surfaces, a closed distal end and an opening at its proximal end located above the upper surface of the base member;
wherein said pouch is configured to receive the glans portion of a user's penis therein with the ventral side of the shaft of the user's penis located adjacent the upper surface of the base member.

2. The male incontinence device of claim 1 wherein each of the base member and the pouch comprise a fluid impermeable layer, a fluid permeable layer, and an absorbent core located therebetween.

3. The male incontinence device of claim 2, wherein the upper surface of the base member comprises said fluid permeable layer, the lower surface of the base member comprises said fluid impermeable layer, the upper and lower interior surfaces of the pouch comprise said fluid permeable layer, and the upper and lower exterior surfaces of the pouch comprise said fluid impermeable layer.

4. The male incontinence device of claim 3, wherein said base member and said pouch comprise a unitary, multi-layer laminate structure.

5. The male incontinence device of claim 4, wherein said pouch comprises a lower pouch section extending away from the distal end of the base member, and an upper pouch section extending away from the lower pouch section, wherein the upper pouch section is folded over the lower pouch section with the fold between the upper and lower pouch sections defining the closed distal end of the pouch.

6. The male incontinence device of claim 5, wherein said upper and lower pouch sections each have opposing sides located on opposite sides of the pouch, further wherein said upper and lower pouch sections are sealed to one other along their opposing sides.

7. The male incontinence device of claim 1, further comprising an elastic strap extending across the width of the device adjacent the opening of said pouch, wherein said elastic strap is configured to press the ventral side of the shaft of the user's penis against the upper surface of the base member so as to retain the glans portion of the user's penis within said pouch.

8. The male incontinence device of claim 6, wherein the opening of said pouch is wider than the distal end of said base member.

9. The male incontinence device of claim 6, wherein said pouch tapers in width from the pouch opening towards the distal end of the pouch.

10. The male incontinence device of claim 2, wherein at least said pouch further comprises an ADL located between the fluid permeable layer and the absorbent core of said pouch.

11. The male incontinence device of claim 10, wherein the ADL is located between the fluid permeable layer and the absorbent core of said pouch and said base member.

12. The male incontinence device of claim 1, further comprising an adhesive layer located on a portion of the lower surface of the base member, along with a removable release sheet covering said adhesive layer prior to use.

13. The male incontinence device of claim 12, wherein said adhesive layer extends along a portion of the lower surface of the base member and a portion of the lower exterior surface of said pouch.

14. A method of fabricating the male incontinence device of claim 6, comprising:
(a) providing said multi-layer laminate structure comprising said a fluid impermeable layer, said fluid permeable layer, and said absorbent core located therebetween, said laminate structure in the form of the base member, the lower pouch section extending away from the distal end of the base member, and the upper pouch section extending away from the lower pouch section;
(b) folding the upper pouch section over the lower pouch section with the fold between the upper and lower pouch sections defining the closed distal end of the pouch, so as to form said pouch; and
(c) sealing said upper and lower pouch sections to one other along their opposing sides.
